(19) United States
(12) Patent Application Publication
(10) Pub. No.: US 2023/0201537 A1
(43) Pub. Date: Jun. 29, 2023

(54) Introducer Sheaths for Introducer Needles and Methods Thereof

(71) Applicant: Bard Access Systems, Inc., Salt Lake City, UT (US)

(72) Inventors: Glade H. Howell, Draper, UT (US); Daniel B. Blanchard, Bountiful, UT (US); Eric W. Lindekugel, Salt Lake City, UT (US); Kyle G. Thornley, Farmington, UT (US); Joe Spataro, Cottonwood Heights, UT (US)

(21) Appl. No.: 18/086,517

(22) Filed: Dec. 21, 2022

Related U.S. Application Data
(60) Provisional application No. 63/294,737, filed on Dec. 29, 2021.

(51) Int. Cl. A61M 25/06; A61M 25/00

(52) U.S. Cl. CPC A61M 25/0606; A61M 25/0618; A61M 25/0097; A61M 25/0693

(57) ABSTRACT

Introducer needles can be used with rapidly insertable central catheters (“RICCs”) or even other central venous catheters (“CVCs”). An introducer needle can include a needle shaft, a sheath, and a needle hub. The needle shaft can include a needle slot extending from a proximal portion of the needle shaft through a distal needle tip. The sheath can be disposed over the needle shaft and fixed in location thereon. The sheath can seal the needle slot under the sheath but for a sheath opening in a proximal portion of the sheath. The needle hub can be around at least the proximal portion of the needle shaft. The introducer needle can be made by creating the needle slot in the needle shaft such as by cutting or grinding the needle slot therein, disposing the sheath over the needle shaft, and fixing the sheath on the needle shaft.

### PRIORITY

This application claims the benefit of priority to U.S. Provisional Application No. 63/294,737, filed Dec. 29, 2021, which is incorporated by reference in its entirety into this application.

### BACKGROUND

Central venous catheters (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are introducer sheaths for introducer needles and methods thereof, which introducer needles can be used separately or together with rapidly insertable central catheters (“RICCs”) to address the forgoing need.

### SUMMARY

Disclosed herein an introducer needle including, in some embodiments, a needle shaft, a sheath, and a needle hub. The needle shaft includes a needle slot extending from a proximal portion of the needle shaft through a distal needle tip. The sheath is disposed over the needle shaft and fixed in at least location thereon. The sheath seals the needle slot under the sheath but for a sheath opening in a proximal portion of the sheath. The needle hub is around at least the proximal portion of the needle shaft.

In some embodiments, the sheath is adhered to the needle shaft with an intervening adhesive.

In some embodiments, the adhesive is a pressure-sensitive adhesive.

In some embodiments, the adhesive is a cured epoxy resin.

In some embodiments, the adhesive is a cured tie-layer resin.

In some embodiments, the sheath is laser-welded to the needle shaft. The sheath is translucent to at least a wavelength or range of wavelengths of a laser used to laser-weld the sheath to the needle shaft.

In some embodiments, the sheath is translucent to visible light. The sheath being translucent to visible light enables a user to witness blood flash into the needle shaft through the sheath sealing the needle slot thereunder upon performing a percutaneous puncture with the introducer needle.

In some embodiments, the needle shaft includes one or more outwardly protruding protrusions. The one-or-more protrusions are in the proximal portion of needle shaft proximal of the needle slot, the proximal portion of needle shaft distal of the needle slot, a distal portion of needle shaft proximal of the needle tip, or a combination thereof. The one-or-more protrusions are configured to restrict the sheath from sliding over the needle shaft.

In some embodiments, the one-or-more protrusions are one or more pillars, respectively.

In some embodiments, the one-or-more protrusions are one or more arcuate ridges, respectively. Each arcuate ridge of the one-or-more arcuate ridges is along at least a portion of a circumference of the needle shaft.

In some embodiments, the sheath includes one or more through holes shaped in accordance with the one-or-more protrusions. The one-or-more protrusions of the needle shaft are disposed in the one-or-more through holes of the sheath, respectively.

In some embodiments, the proximal portion of the needle shaft proximal of the needle slot includes a flared portion configured to restrict the sheath from proximally sliding over the needle shaft.

In some embodiments, the proximal portion of the needle shaft proximal of the needle slot includes a stepped portion configured to restrict the sheath from proximally sliding over the needle shaft.

In some embodiments, the needle shaft includes a recessed portion between the proximal portion of the needle shaft and the needle tip configured to restrict the sheath from proximally or distally sliding over the needle shaft. The sheath is disposed in the recessed portion of the needle shaft flush with a remainder of the needle shaft.

In some embodiments, the sheath includes an inwardly protruding protrusion in the proximal portion of sheath configured sit within the needle slot and abut a proximal end of the needle slot to restrict the sheath from proximally sliding over the needle shaft.

In some embodiments, the sheath includes an inwardly protruding longitudinal ridge extending from the proximal portion of sheath to a distal portion of the sheath. The ridge is configured sit within the needle slot and abut a proximal end of the needle slot to restrict the sheath from proximally sliding over the needle shaft.

In some embodiments, the sheath is disposed over the needle shaft with an engineering fit selected from a transition fit and an interference fit.

In some embodiments, an outer surface of the needle shaft is textured or

roughened.

In some embodiments, the sheath has a sheath-wall thickness from about 0.001″ to about 0.003″.

In some embodiments, the sheath has a sheath-wall thickness of about 0.003″ to about 0.006″.

In some embodiments, the sheath has a sheath-wall thickness from about 0.006″ to about 0.008″.

Also disclosed herein is an introducer needle including, in some embodiments, a needle shaft, a longitudinal strip, and a needle hub. The needle shaft includes a needle slot extending from a proximal portion of the needle shaft through a distal needle tip. The strip is adhered to the needle shaft sealing the needle slot thereunder but for a proximal portion of the needle slot, thereby creating an opening in a side of the introducer needle. The needle hub is around at least the proximal portion of the needle shaft.

Also disclosed herein is a method of making an introducer needle. The method includes, in some embodiments, a needle slot-creating step, a sheath-disposing step, a sheath-fixing step, and a needle hub-fixing step. The needle slot-creating step includes creating a needle slot in a needle shaft. The needle slot extends from a proximal portion of the needle shaft through a distal needle tip. The sheath-disposing step includes disposing a sheath over the needle shaft. The sheath-fixing step includes fixing the sheath to the needle shaft. The sheath seals the needle slot under the sheath. The needle hub-fixing step includes fixing a needle hub around at least the proximal portion of the needle shaft.

In some embodiments, the needle slot-creating step includes cutting or grinding the needle slot into the needle shaft.

In some embodiments, the sheath-disposing step includes inserting the needle shaft into the sheath.

In some embodiments, the sheath-fixing step includes adhering the sheath to the needle shaft with an adhesive selected from a pressure-sensitive adhesive, an epoxy, and a tie-layer resin.

In some embodiments, the sheath-fixing step includes laser-welding the sheath to the needle shaft. The sheath is translucent to at least a wavelength or range of wavelengths of a laser used for the laser-welding.

In some embodiments, the sheath-fixing step includes welding one or more outwardly protruding protrusions onto the needle shaft, cutting one or more through holes into the sheath, and disposing the sheath over the needle shaft such that the one-or-more protrusions are disposed in the one-or-more through holes, respectively.

In some embodiments, the sheath-fixing step includes recessing a portion of the needle shaft between the proximal portion of the needle shaft and the needle tip to create a recessed portion of the needle shaft and subsequently disposing the sheath over the needle shaft in the recessed portion of the needle shaft such that the sheath is flush with a remainder of the needle shaft.

In some embodiments, the method further includes a sheath-opening creating step. The sheath-opening creating step includes creating a sheath opening in the sheath by way of laser cutting after the sheath-disposing step. The sheath seals the needle slot under the sheath but for the sheath opening in a proximal portion of the sheath.

These and other features of the concepts provided herein will become more apparent to those of skill in the art in view of the accompanying drawings and following description, which describe particular embodiments of such concepts in greater detail.

### DRAWINGS

FIG. 1 illustrates a RICC insertion assembly in accordance with some embodiments.

FIG. 2 illustrates a sealing module of a coupler of the RICC insertion assembly in accordance with some embodiments.

FIG. 3 illustrates a RICC of the RICC insertion assembly in accordance with some embodiments.

FIG. 4 illustrates a detailed view of a distal portion of a catheter tube of the RICC in accordance with some embodiments.

FIG. 5 illustrates a transverse cross section of the distal portion of the catheter tube in accordance with some embodiments.

FIG. 6 illustrates another transverse cross section of the distal portion of the catheter tube in accordance with some embodiments.

FIG. 7 illustrates a longitudinal cross section of the distal portion of the catheter tube in accordance with some embodiments.

FIG. 8 illustrates a top view of the introducer needle in accordance with some embodiments.

FIG. 9 illustrates a sheath of the introducer needle in accordance with some embodiments.

FIG. 10 illustrates a needle shaft of the introducer needle with a needle slot extending along at least a portion of a top of the needle shaft in accordance with some embodiments.

FIG. 11 illustrates a detailed view of a distal portion of the introducer needle with the sheath adhered to the needle shaft with an intervening adhesive in accordance with some embodiments.

FIG. 12 illustrates a transverse cross section of a proximal portion of the introducer needle with the sheath adhered to the needle shaft with the adhesive in accordance with some embodiments.

FIG. 13 illustrates a longitudinal cross section of the distal portion of the introducer needle with the sheath adhered to the needle shaft with the adhesive in accordance with some embodiments.

FIG. 14 illustrates a detailed view of the distal portion of the introducer needle with the sheath laser-welded to the needle shaft in accordance with some embodiments.

FIG. 15 illustrates a detailed view of the needle shaft with one or more outwardly protruding protrusions in the proximal portion of needle shaft in accordance with some embodiments.

FIG. 16 illustrates a detailed view of the proximal portion of the introducer needle with the needle shaft including a stepped portion in accordance with some embodiments.

FIG. 17 illustrates the introducer needle with the needle shaft including a recessed portion in accordance with some embodiments.

FIG. 18 illustrates the introducer needle with the needle shaft including a flared portion in accordance with some embodiments.

FIG. 19 illustrates a detailed view of the proximal portion of the introducer needle with the needle shaft including a flared portion in accordance with some embodiments.

FIG. 20 illustrates a detailed view of the proximal portion of the introducer needle with the sheath including an inwardly protruding protrusion or ridge sitting in the needle slot of the needle shaft in accordance with some embodiments.

FIG. 21 illustrates a transverse cross section of the introducer needle with the sheath including the inwardly protruding protrusion or ridge sitting in the needle slot of the needle shaft in accordance with some embodiments.

FIG. 22 illustrates a detailed view of the distal portion of the introducer needle with a relatively thin sheath in accordance with some embodiments.

FIG. 23 illustrates a distal end-on view of the introducer needle with the relatively thin sheath in accordance with some embodiments.

FIG. 24 illustrates a detailed view of the distal portion of the introducer needle with a relatively thick sheath in accordance with some embodiments.

FIG. 25 illustrates a distal end-on view of the introducer needle with the relatively thick sheath in accordance with some embodiments.

FIG. 26 illustrates a detailed view of the distal portion of the introducer needle with a longitudinal strip adhered to the needle shaft over the needle slot in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above with respect to the Seldinger technique, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are introducer sheaths for introducer needles, which introducer needles can be used separately or together with RICCs or even other CVCs. For example, an introducer needle can include a needle shaft, a sheath, and a needle hub. The needle shaft can include a needle slot extending from a proximal portion of the needle shaft through a distal needle tip. The sheath can be disposed over the needle shaft and fixed in at least location thereon. The sheath can seal the needle slot under the sheath but for a sheath opening in a proximal portion of the sheath. The needle hub can be around at least the proximal portion of the needle shaft. Methods of making the introducer needles are disclosed as well.

The foregoing features as well as other features of the introducer sheaths, the introducer needles, the RICCs, and the methods disclosed herein will become more apparent to those of skill in the art in view of the accompanying drawings and following description, which describe particular embodiments of the foregoing in greater detail beginning with RICC insertion assemblies. However, it should be understood the RICCs of the RICC insertion assemblies are but one type of catheter that can be used together with the introducer needles and incorporated into catheter insertion assemblies like those disclosed herein. Indeed, other CVCs, peripherally inserted central catheters (“PICCs”), dialysis catheters, or the like can also be used together with the introducer needles and incorporated into catheter insertion assemblies and used.

### RICC Insertion Assemblies

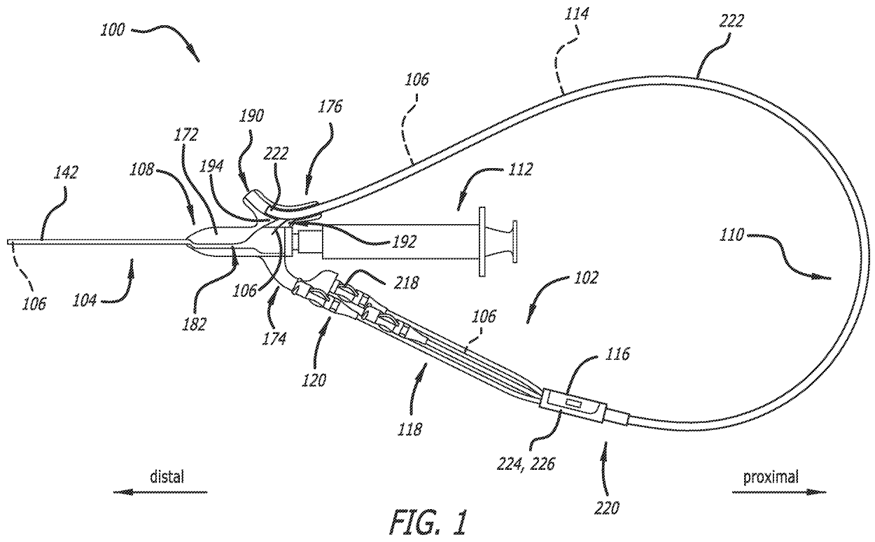

FIG. 1 illustrates a RICC insertion assembly 100 in accordance with some embodiments.

As shown, the RICC insertion assembly 100 includes a RICC 102, an introducer needle 104, an access guidewire 106, and a coupler 108 coupling the RICC 102, the introducer needle 104, and the access guidewire 106 together in a ready-to-operate state of the RICC insertion assembly 100. As set forth in more detail below, the proximal end of the access guidewire 106 is coupled to the coupler 108 and the distal end of the access guidewire 106 is disposed in the needle lumen 158 of the introducer needle 104 in the ready-to-operate state of the RICC insertion assembly 100. This enforces a loop 110 in the access guidewire 106, which loop 110 the RICC 102 is disposed over in the ready-to-operate state of the RICC insertion assembly 100 keeping the RICC insertion assembly 100 in a relatively compact form.

The RICC insertion assembly 100 can further include a syringe 112 fluidly coupled to the introducer needle 104 in the ready-to-operate state of the RICC insertion assembly 100. As set forth below, the sealing module of the coupler 108 seals around the proximal portion of the introducer needle 104 and the distal portion of the access guidewire 106 when the sealing-module insert 198 is compressed in the sealing-module cavity 178 of the coupler housing 172 in one or more states of the RICC insertion assembly 100. In particular, the sealing module seals over the sheath opening 162 of the sheath 142 that opens to the needle slot 150 of the needle shaft 144. Outside of the sealing module, the sheath 142 seals the needle slot 150 of the needle shaft 144. Such seals enable the syringe 112 to aspirate blood.

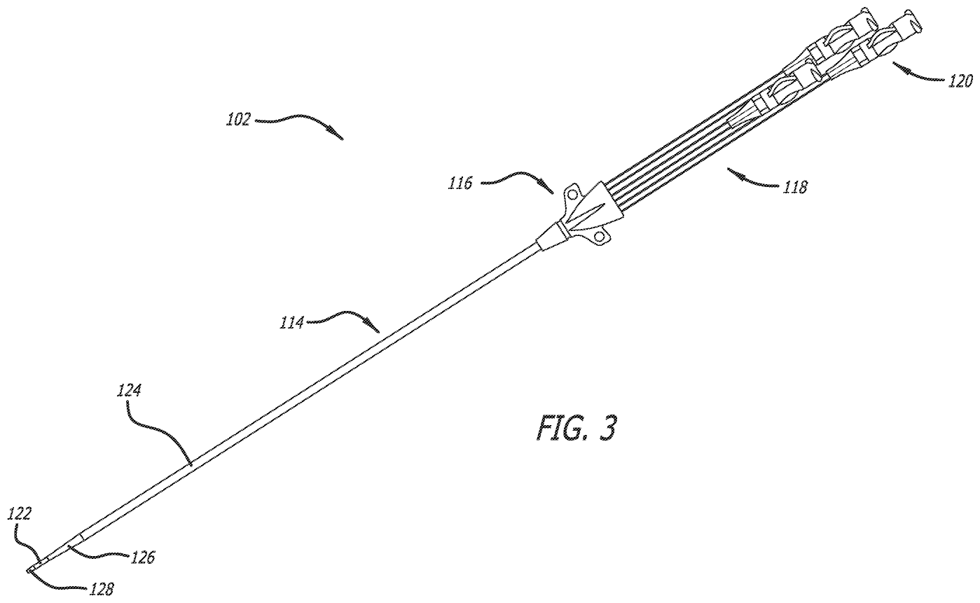

FIG. 3 illustrates the RICC 102 of the RICC insertion assembly 100 in accordance with some embodiments.

As shown, the RICC 102 includes a catheter tube 114, a catheter hub 116, one or more extension legs 118, and one or more extension-leg connectors 120.

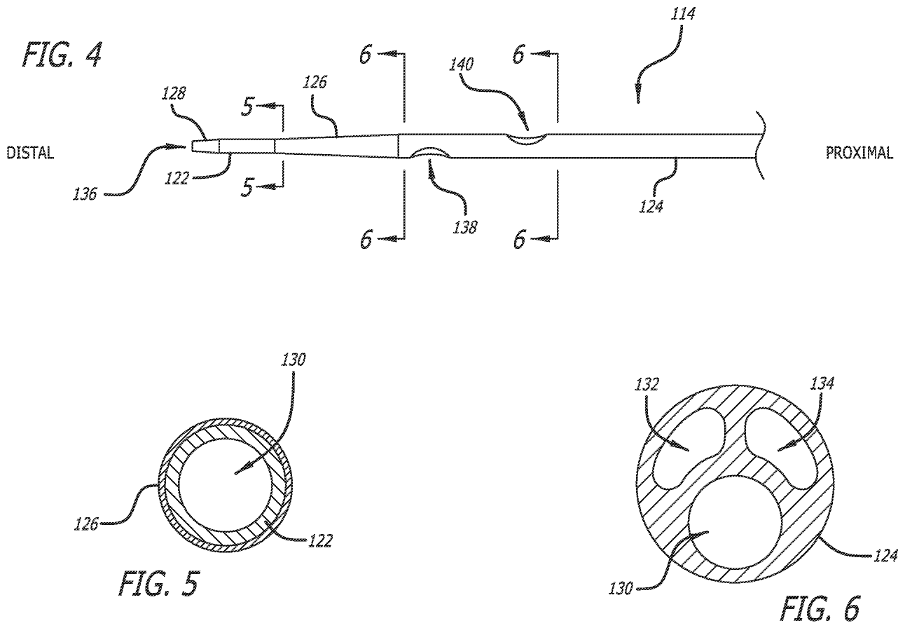

FIGS. 4-7 illustrate various views of the catheter tube 114 of the RICC 102 in accordance with some embodiments.

The catheter tube 114 includes a first section 122 in a distal portion of the catheter tube 114, a second section 124 in the distal portion of the catheter tube 114 proximal of the first section 122, and a tapered junction 126 between the first and second sections 122 and 124 of the catheter tube 114.

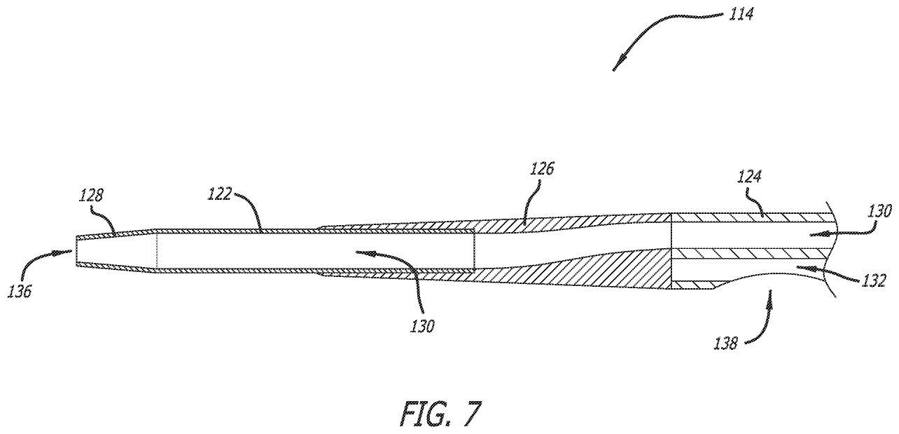

The first section 122 of the catheter tube 114 includes a catheter tip 128 having a relatively short taper from an outer diameter of a distal portion of the first section 122 distal of the junction 126 to an outer diameter of a distal end of the first section 122. The taper of the catheter tip 128 is configured for immediate dilation of tissue about a needle tract established with the introducer needle 104 up to the outer diameter of the distal portion of the first section 122 of the catheter tube 114. As best shown in FIG. 7, the first section 122 of the catheter tube 114 also includes a proximal portion disposed in a bore of a distal portion of the junction 126 and fixedly coupled thereto such as by a solvent bond, an adhesive bond, or a heat weld.

The second section 124 of the catheter tube 114 includes a consistent outer diameter over its length from a distal end of the second section 124 to a proximal end of the second section 124. The consistent diameter of the second section 124 of the catheter tube 114 is configured for smooth insertion into the needle tract and targeted vasculature subsequent to any dilation by the first section 122 of the catheter tube 114 and the junction 126. The distal end of the second section 124 of the catheter tube 114 has a flat face flush with the flat-faced proximal end of the junction 126 and fixedly coupled thereto such as by a solvent bond, an adhesive bond, or a heat weld.

The junction 126 includes a taper over its length from a proximal end of the junction 126 to a distal end of the junction 126. The taper of the junction 126 is configured for immediate dilation of the tissue about the needle tract from the outer diameter of the proximal portion of the first section 122 of the catheter tube 114 to the outer diameter of the second section 124 of the catheter tube 114. An abluminal surface of the junction 126 smoothly transitions from an abluminal surface of the first section 122 of the catheter tube 114 to an abluminal surface of the second section 124 of the catheter tube 114 without edges that catch on skin when the catheter tube 114 is inserted into the needle tract. In addition to the edges being minimal to negligible, the edges can include solvent-interdiffused polymeric material of the polymeric materials from which the catheter tube 114 is formed, which smoothens the transitions from the first section 122 of the catheter tube 114 to the junction 126 and from the junction 126 to the second section 124 of the catheter tube 114. Notably, the junction 126 has a length approximately commensurate with a length of an exposed portion of the first section 122 of the catheter tube 114 or between lengths of exposed portions of the first and second sections 122 and 124 of the catheter tube 114. As such, the length of the exposed portion of the first section 122 of the catheter tube 114 is less than the length of the junction 126 up to approximately commensurate with the length of the junction 126.

The first section 122 of the catheter tube 114 is formed of a first polymeric material (e.g., a polytetrafluoroethylene, a polypropylene, or a polyurethane) having a first durometer. The second section 124 of the catheter tube 114 is formed of a second polymeric material (e.g., a polyvinyl chloride, a polyethylene, another polyurethane, or a silicone) having a second durometer less than the first durometer. For example, the first section 122 of the catheter tube 114 can be formed of a first polyurethane having the first durometer while the second section 124 of the catheter tube 114 can be formed of a second, different polyurethane (e.g., a same or different diisocyanate or triisocyanate reacted with a different diol or triol, a different diisocyanate or triisocyanate reacted with a same or different diol or triol, a same diisocyanate or triisocyanate reacted with a same diol or triol under different conditions or with different additives, etc.) having the second durometer less than the first durometer. Indeed, polyurethanes are advantageous for the catheter tube 114 in that polyurethanes can be relatively rigid at room-temperature but become more flexible in vivo at body temperature, which reduces irritation to vessel walls as well as phlebitis. Polyurethanes are also advantageous in that they can be less thrombogenic than some other polymers. The junction 126 is formed of the second polymeric material or a third polymeric material (e.g., yet another polyurethane) having a third durometer less than the first durometer and greater than, approximately equal to, or less than the second durometer.

It should be understood the first durometer of the first polymeric material, the second durometer of the second polymeric material, and the third durometer of the third polymeric material can be on different scales (e.g., Type A or Type D). With this understanding, the second durometer of the second polymeric material or the third durometer of the third polymeric material might not be numerically less than the first durometer of the first polymeric material when the second durometer or the third durometer is less than the first durometer. Indeed, the hardness of the second polymeric material or the third polymeric material can still be less than the hardness of the first polymeric material as the different scales—each of which ranges from 0 to 100—are designed for characterizing different materials in groups of the materials having a like hardness.

In accordance with the first section 122 of the catheter tube 114, the second section 124 of the catheter tube 114, and the junction 126 between the first and second sections 122 and 124 of the catheter tube 114 set forth above, the catheter tube 114 possesses a column strength, optionally, in combination with the access guidewire 106, sufficient to prevent buckling of the catheter tube 114 when inserted into a needle tract established by with the introducer needle 104. The column strength of the catheter tube 114 is also sufficient to prevent buckling of the catheter tube 114 when advanced through a vasculature of a patient without dilation of tissue about the needle tract or any blood vessels of the vasculature beforehand with a separate dilator.

The catheter tube 114 includes one or more catheter-tube lumens extending through the catheter tube 114; however, only one catheter-tube lumen typically extends from a proximal end of the catheter tube 114 to a distal end of the catheter tube 114 in a multiluminal RICC (e.g., a diluminal RICC, a triluminal RICC, a tetraluminal RICC, a pentaluminal RICC, a hexaluminal RICC, etc.). (See FIGS. 4-7.) Indeed, the first section 122 of the catheter tube 114 typically includes a single lumen therethrough as shown in FIGS. 4 and 7.

The catheter hub 116 is coupled to a proximal portion of the catheter tube 114. The catheter hub 116 includes one or more catheter-hub lumens corresponding in number to the one-or-more catheter-tube lumens. The one-or-more catheter-hub lumens extends through an entirety of the catheter hub 116 from a proximal end of the catheter hub 116 to a distal end of the catheter hub 116.

Each extension leg of the one-or-more extension legs 118 is coupled to the catheter hub 116 by a distal portion thereof. The one-or-more extension legs 118 respectively include one or more extension-leg lumens, which, in turn, correspond in number to the one-or-more catheter-hub lumens. Each extension-leg lumen of the one-or-more extension-leg lumens extends through an entirety of the extension leg from a proximal end of the extension leg to a distal end of the extension leg.

Each extension-leg connector of the one-or-more extension-leg connectors 120 is over a proximal portion of an extension leg of the one-or-more extension legs 118. For example, each extension-leg connector of the one-or-more extension-leg connectors 120 can be a Luer connector over a proximal portion of an extension leg of the one-or-more extension legs 118. Through such an extension-leg connector, a corresponding extension leg and the extension-leg lumen thereof can be connected to another medical device and a lumen thereof. However, in the ready-to-operate state of the RICC insertion assembly 100 at least one extension-leg connector (e.g., the extension-leg connector including part of the primary lumen 130 of the RICC 102) is indirectly connected via the intervening access-guidewire hub 218 to the access guidewire-connecting side arm 174 of the coupler 108 to enforce the loop 110 in the access guidewire 106 and the RICC 102 thereover.

As shown, the RICC 102 is a triluminal RICC including a set of three lumens; however, the RICC 102 is not limited to the set of the three lumens as set forth above. The set of three lumens includes a primary lumen 130, a secondary lumen 132, and a tertiary lumen 134 formed of fluidly connected portions of three catheter-tube lumens, three catheter-hub lumens, and three extension-leg lumens. The primary lumen 130 has a primary-lumen aperture 136 in the distal end of the first section 122 of the catheter tube 114, which corresponds to the distal end of the catheter tube 114 and a distal end of the RICC 102. The secondary lumen 132 has a secondary-lumen aperture 138 in a side of the distal portion of the catheter tube 114. The tertiary lumen 134 has a tertiary-lumen aperture 140 in the side of the distal portion of the catheter tube 114 proximal of the secondary-lumen aperture 138.

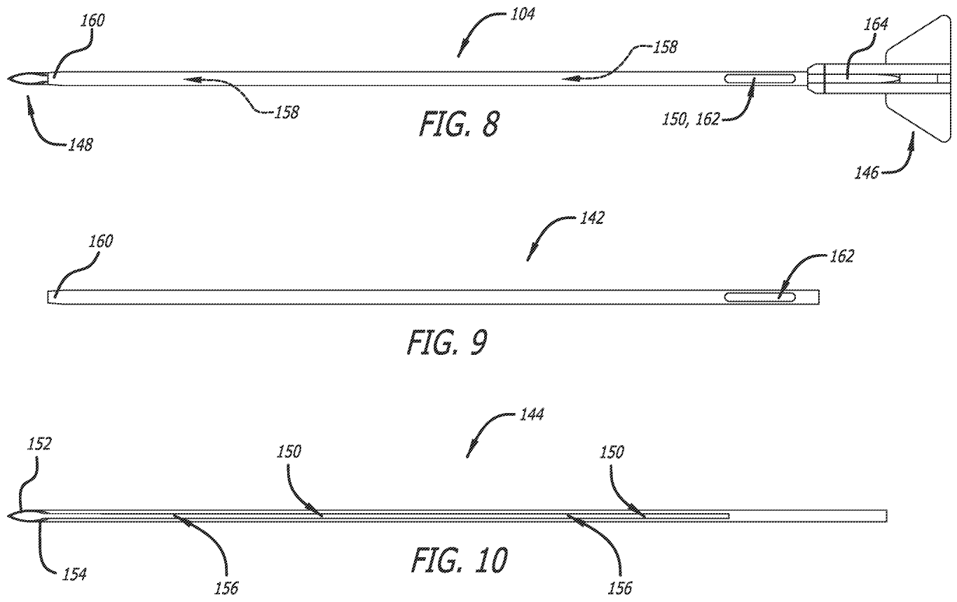

FIG. 8 illustrates a top view of the introducer needle 104 of the RICC insertion assembly 100 in accordance with some embodiments. FIG. 9 illustrates a sheath 142 of the introducer needle 104 in accordance with some embodiments. FIG. 10 illustrates a needle shaft 144 of the introducer needle 104 in accordance with some embodiments.

As shown, the introducer needle 104 includes the needle shaft 144, the sheath 142 over the needle shaft 144, and a needle hub 146 in a proximal portion of the introducer needle 104 over or around at least a proximal portion of the needle shaft 144 including a proximal end of the needle shaft 144. In at least the ready-to-operate state of the RICC insertion assembly 100, the needle shaft 144 and the sheath 142 extend from the needle hub 146, through the sealing module of the coupler 108, and out a distal end of the coupler housing 172.

The needle shaft 144 includes a needle tip 148 in a distal portion of the needle shaft 144 and a longitudinal needle slot 150 extending from the proximal portion of the needle shaft 144 through the needle tip 148.

The needle tip 148 includes a bevel having a tip or secondary bevel 152 and a primary bevel 154 proximal of the tip bevel 152, wherein the primary bevel 154 terminates in a proximal heel. A tip-bevel angle of the tip bevel 152 is greater than a primary-bevel angle of the primary bevel 154 such that the bevel provides a smooth transition over the needle tip 148. Such a needle tip is thusly configured for establishing a needle tract from an area of skin into a blood-vessel lumen of a patient. Notably, a top of the needle shaft 144 includes the heel of the bevel, a bottom of the needle shaft 144 includes the tip bevel 152 of the bevel, and a side of the needle shaft 144 is between the heel and tip bevel 152 of the bevel.

The needle slot 150 extends from the proximal portion of the needle shaft 144 through the needle tip 148, thereby forming a needle channel 156 along a majority of a length of the needle shaft 144 as opposed to a needle lumen therethrough.

The needle slot 150 can linearly extend along at least a portion of the top of the needle shaft 144 such as a minority of the top of the needle shaft 144 up to a majority of the top of the needle shaft 144 but short (e.g., by about 0.2-0.3″) of a proximal end of the needle shaft 144. The needle slot 150 can even extend along an entirety of the top of the needle shaft 144 including the proximal end of the needle shaft 144. As shown in FIG. 10, for example, the needle slot 150 extends along the majority of the top of the needle shaft 144 from the proximal portion of the needle shaft 144 through the needle tip 148, short of the proximal end of the needle shaft 144 and bisecting the heel of the bevel. When the needle slot 150 bisects the heel of the bevel, the flexural strength of the needle shaft 144 is greater along the top and bottom of the needle shaft 144 about at least the distal portion of the needle shaft 144 when compared to, for example, a needle slot in the side of the needle shaft 144, which configuration is also possible. This can be important because the introducer needle 104 is typically flexed into the top or bottom of the needle shaft 144 about the distal portion of the needle shaft 144 during a percutaneous puncture with the introducer needle 104 when establishing a needle tract from an area of skin to a blood-vessel lumen of a patient.

The needle slot 150 has a needle-slot width sized in accordance with at least an outer diameter of the access guidewire 106, which allows the access guidewire 106 to pass from the proximal portion of the needle shaft 144 through the needle tip 148 when the introducer needle is withdrawn following a percutaneous puncture therewith. Indeed, the needle slot 150 can have a constant needle-slot width sized in accordance with at least an outer diameter of the access guidewire 106 such as slightly larger than an outer diameter of the access guidewire 106. For example, the slot width can range from about 0.018″ to about 0.035″.

Additional description for the needle shaft 144 including the one-or-more protrusions 230 in the proximal portion of needle shaft 144, the flared portion 234 of the needle shaft 144, the stepped portion 236 of the needle shaft 144, and the recessed portion 238 of the needle shaft 144 is set forth below with respect to the various fixing means for fixing the sheath 142 to the needle shaft 144 to prevent proximal or distal movement of the sheath 142 relative to the needle shaft 144.

Notably, the needle shaft 144 includes the needle channel 156, whereas the introducer needle 104 includes a needle lumen 158. This is because the needle lumen 158 results from the combination of the needle shaft 144 and the sheath 142 with the sheath 142 over the needle shaft 144. Indeed, the sheath 142 over the needle shaft 144 seals the needle channel 156 forming the needle lumen 158 of the introducer needle 104, thereby enabling the syringe 112 to aspirate blood following a percutaneous puncture with the introducer needle 104 that establishes a needle tract from an area of skin to a blood-vessel lumen of a patient.

The sheath 142 includes a sheath tip 160 in a distal portion of the sheath 142 and a sheath opening 162 in a side of the proximal portion of the sheath 142.

The sheath tip 160 includes a relatively short taper from an outer diameter of the distal portion of the sheath 142 to an outer diameter of a distal end of the sheath 142, the latter of which is commensurate with an outer diameter of the distal portion of the needle shaft 144 (excepting the needle shaft 144 set forth below with the recessed portion thereof). The taper has a taper angle less than the primary-bevel angle of the primary bevel 154 of the needle tip 148, which, in turn, is less than the tip-bevel angle of the tip bevel 152 of the needle tip 148. The sheath tip 160 including such a taper is configured to provide a smooth transition from the needle tip 148 to the sheath body during percutaneous puncture to establish a needle tract from an area of skin to a blood-vessel lumen of a patient.

The sheath opening 162 opens to the needle slot 150 of the needle shaft 144 allowing the access guidewire 106 to pass through the sheath opening 162 and into the needle slot 150 in the ready-to-operate state of the RICC insertion assembly 100. Thus, the sheath opening 162 has a width approximately commensurate with a width of the needle slot 150 thereunder, which needle slot 150, in turn, is sized in accordance with the diameter of the access guidewire 106. The sheath opening 162 also has a length sufficient to allow the access guidewire 106 to pass through the sheath opening 162 and into the needle slot 150. Notably, the sheath 142 over the needle shaft 144 seals the needle slot 150 thereunder except for that under the sheath opening 162. However, the sealing module of the coupler 108 seals over the needle slot 150 exposed by the sheath opening 162 by sealing the proximal portions of the needle shaft 144 and the sheath 142 therein, thereby enabling the syringe 112 to aspirate blood following a percutaneous puncture with the introducer needle 104 that establishes a needle tract from an area of skin to a blood-vessel lumen of a patient.

The sheath 142 can be a cuttable or a splittable sheath configured for respectively cutting or splitting the sheath 142 away from the needle shaft 144 to allow the access guidewire 106 to escape from the needle shaft 144 by way of the needle slot 150. When configured to be cut away from the needle shaft 144, the sheath 142 can be formed of a polymeric material such as polyurethane that facilitates the cutting of the sheath 142 away from the needle shaft 144. When configured to be split away from the needle shaft 144, the sheath 142 can include one or more weakened portions (e.g., a longitudinal pattern of perforations, longitudinal grooves, etc.) of the sheath 142 that facilitate the splitting of the sheath 142 away from the needle shaft 144.

The sheath 142, or a sheath body thereof, is formed of a polymeric material configured to facilitate a smooth, consistent insertion of the introducer needle 104 from an area of skin to a blood-vessel lumen of a patient during a percutaneous puncture. In addition, the polymeric material has mechanical properties at a thickness of the sheath 142 (i.e., the sheath-wall thickness set forth below) sufficient to withstand collapse of the sheath 142 into the needle slot 150 of the needle shaft 144 when blood is aspirated through the introducer needle 104 using the syringe 112, notably, while also facilitating the cutting or splitting of the sheath 142 off the needle shaft 144. Such a polymeric material can include, but is not limited to, polyethylene, polypropylene, polyurethane, or polytetrafluoroethylene (“PTFE”). In an example, the sheath 142 can be polyurethane in embodiments of the RICC insertion assembly 100 in which the sheath 142 is cut away from the needle shaft 144. In another example, the sheath 142 can be PTFE or even expanded PTFE (“ePTFE”) in embodiments of the RICC insertion assembly 100 in which the sheath 142 is split away from the needle shaft 144. When the sheath 142 is, for example, ePTFE, the sheath 142 need not include the one-or-more weakened portions of the sheath 142 for the splitting of the sheath 142 away from the needle shaft 144 because ePTFE, itself, facilitates the splitting of the sheath 142 away from the needle shaft 144 on account of the longitudinal arrangement of polymer chains in the ePTFE.

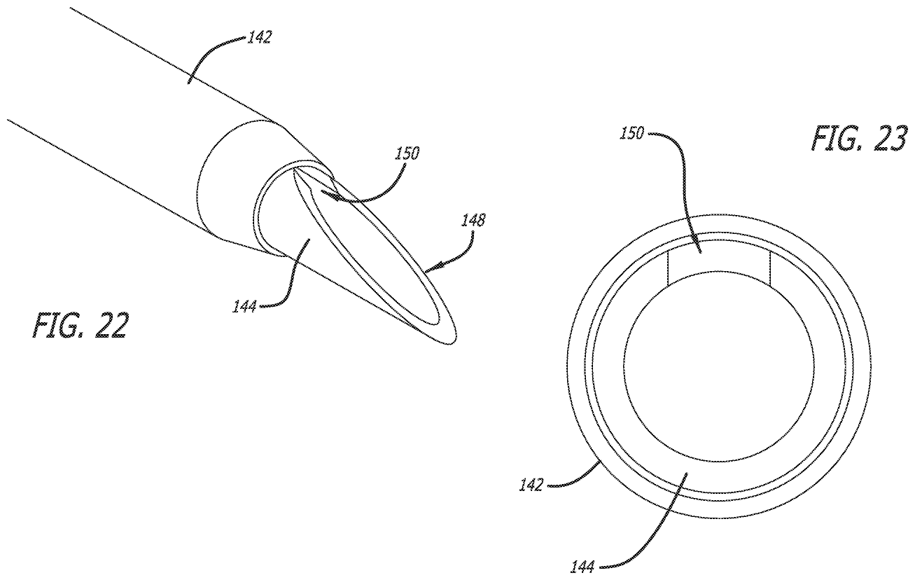

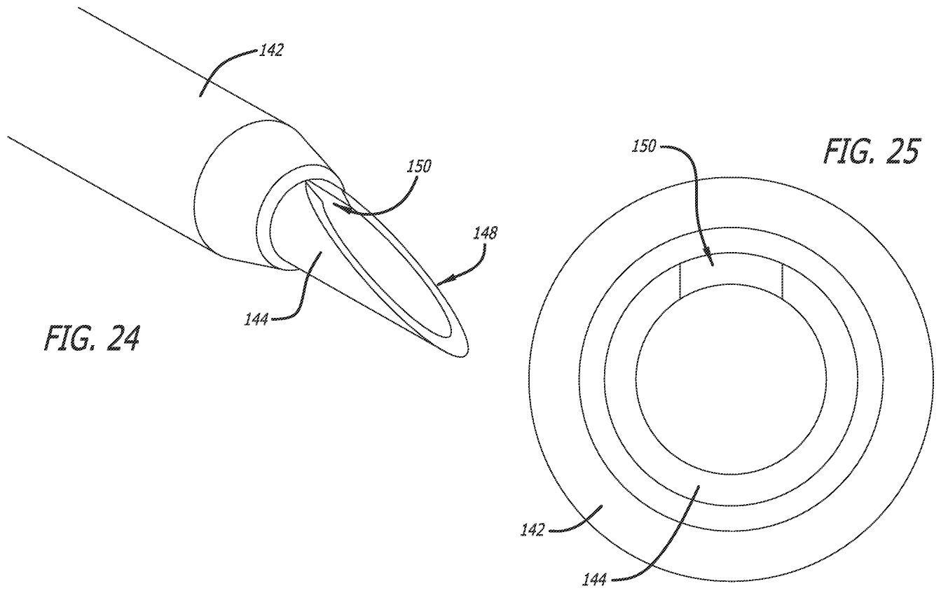

The sheath 142 can have a sheath-wall thickness ranging from about 0.001″ to about 0.010″. In an example, the sheath-wall thickness of the sheath 142 can be relatively thin such as from about 0.001″ to about 0.003″, for example, about 0.001″ as shown in FIGS. 22 and 23. With such a relatively thin sheath 142, a gauge of the introducer needle 104 is relatively small, which results in a narrow needle tract and, thus, minimal tissue damage via dilation when the introducer needle 104 is used to perform a percutaneous puncture. In another example, the sheath-wall thickness of the sheath 142 can be relatively thick such as from about 0.006″ to about 0.008″ as shown in FIGS. 24 and 25. With such a relatively thick sheath 142, the sheath 142 is better able to withstand collapse of the sheath 142 into the needle slot 150 of the needle shaft 144 when blood is aspirated through the introducer needle 104 using the syringe 112. Infusion at relatively high pressures is also possible through the relatively thick sheath 142. In yet another example, the sheath-wall thickness of the sheath 142 can be between the foregoing sheath-wall thicknesses such as from about 0.003″ to about 0.006″.

Additional description for the sheath 142 including translucency of the sheath 142 to one or more wavelengths, the one-or-more through holes 232 of the sheath 142, or the protrusion 240 or ridge 242 of the sheath 142 is set forth below with respect to the various fixing means for fixing the sheath 142 to the needle shaft 144 to prevent proximal or distal movement of the sheath 142 relative to the needle shaft 144.

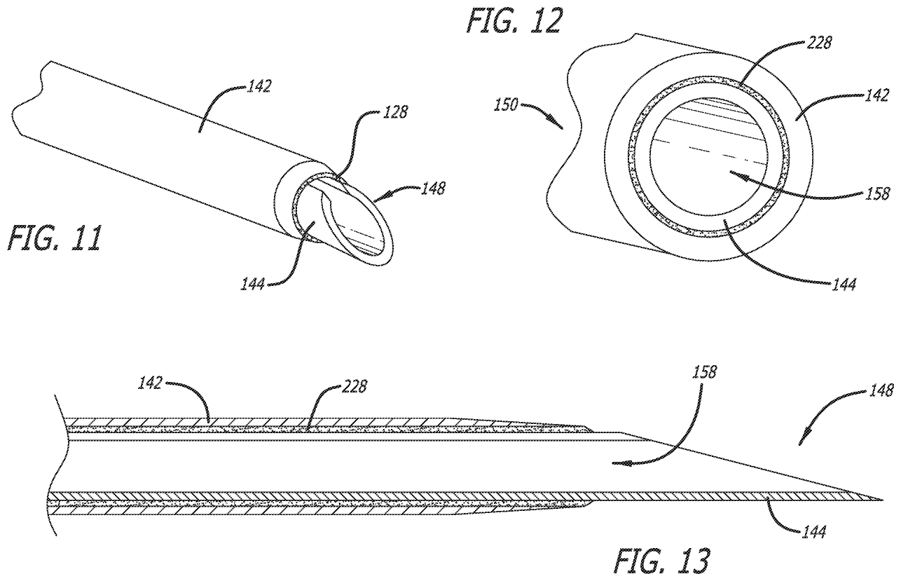

The sheath 142 can be disposed over the needle shaft 144 and fixed in at least location thereto to prevent proximal of the sheath 142, distal movement of the sheath 142, or both proximal and distal movement of the sheath 142 relative to the needle shaft 144. Indeed, any one or more fixing means of the various fixing means for fixing the sheath 142 to the needle shaft 144 shown in FIGS. 11-21 and 26 or set forth below can be used to prevent proximal or distal movement of the sheath 142 relative to the needle shaft 144.

The sheath 142 can be of a heat-shrinkable material with the sheath 142 heat-shrunk over the needle shaft 144, thereby fixing the sheath 142 to the needle shaft 144. The sheath 142 can be fixed to the needle shaft 144 with an engineering fit selected from a transition fit and an interference fit as defined in accordance with the International Organization for Standardization (“ISO”) of Geneva, Switzerland. Notably, the sheath 142 need not be heat-shrunk over the needle shaft 144 to fix the sheath 142 to the needle shaft 144 with such an engineering fit. Indeed, the sheath 142 of any introducer needle 104 disclosed herein can be disposed over the needle shaft 144 and fixed thereto with an engineering fit selected from the transition fit and the interference fit.

FIGS. 11-14 illustrate various views of the introducer needle 104 with the sheath 142 adhered to the needle shaft 144 with an intervening adhesive 228 in accordance with some embodiments.

As shown, the sheath 142 can be adhered to the needle shaft 144 with the adhesive 228, thereby fixing the sheath 142 to the needle shaft 144. Such an adhesive can be a non-reactive adhesive such as a pressure-sensitive adhesive disposed between the sheath 142 and the needle shaft 144, a reactive but reacted adhesive such as an epoxy resin cured between the sheath 142 and the needle shaft 144, or even an intervening tie layer of a cured tie-layer resin (e.g., ethylene-acrylic acid [“EAA”], ethylene methacrylic [“EMAA”], or ethylene-methyl acrylate [“EMA”]) between the sheath 142 and the needle shaft 144. Notably, an outer surface of the needle shaft 144 can be textured or roughened to increase a contact area thereof for interaction with the adhesive 228. However, such a textured or roughened outer surface of the needle shaft 144 need not be limited to embodiments of the introducer needle 104 in which the sheath 142 is adhered to the needle shaft 144. Indeed, the needle shaft 144 of any introducer needle 104 disclosed herein can include the textured or roughened outer surface. For example, asperities in the roughened outer surface of the needle shaft 144 can be useful for biting into an inner or luminal surface of the sheath 142, thereby further fixing the sheath 142 to the needle shaft 144.

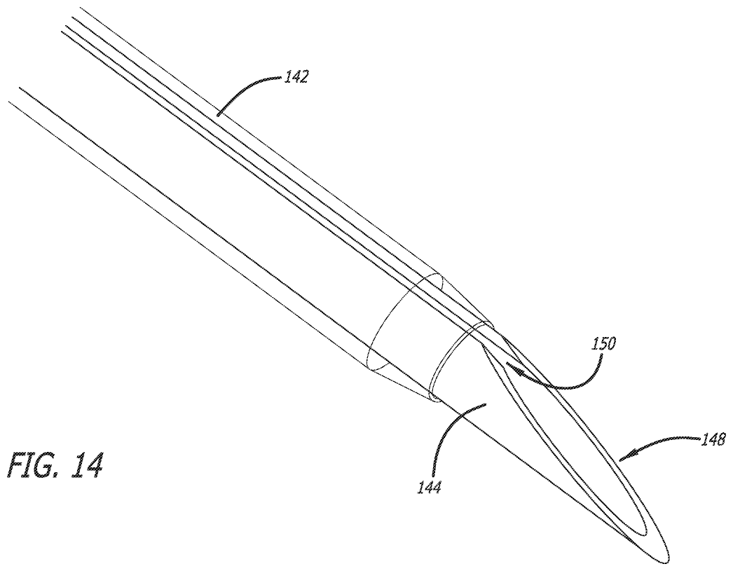

FIG. 14 illustrates a detailed view of a distal portion of the introducer needle 104 with the sheath 142 laser-welded to the needle shaft 144 in accordance with some embodiments.

As shown, the sheath 142 can be laser-welded to the needle shaft 144, thereby fixing the sheath 142 to the needle shaft 144. Such a sheath is translucent to at least a wavelength or range of wavelengths of a laser used to laser-weld the sheath 142 to the needle shaft 144. However, the sheath 142 can also be translucent to visible light. Such a sheath enables a user to witness blood flash into the needle shaft 144 through the sheath 142 sealing the needle slot 150 thereunder upon successfully performing a percutaneous puncture with the introducer needle 104 in which a needle tract is established from an area of skin to a blood-vessel lumen of a patient.

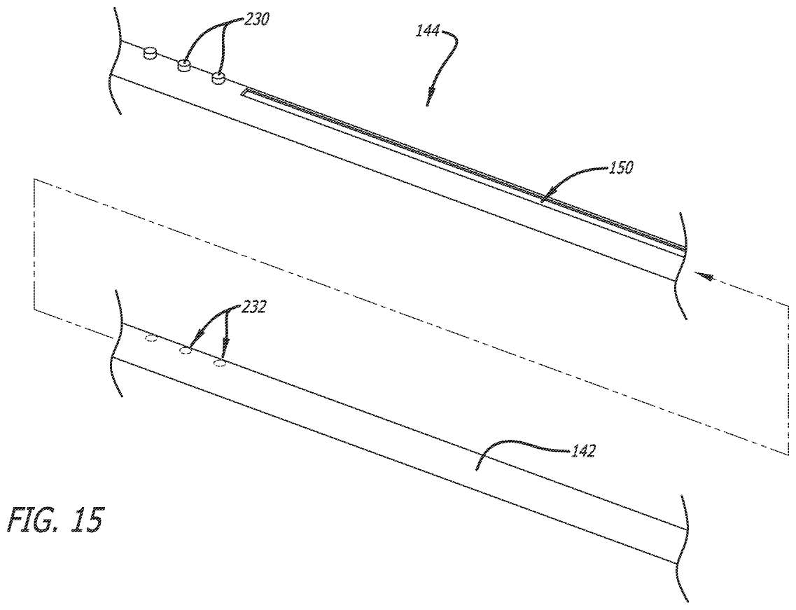

FIG. 15 illustrates a detailed view of the needle shaft 144 with one or more outwardly protruding protrusions 230 and the sheath 142 optionally with one or more through holes 232 shaped in accordance with the one-or-more protrusions 230 in accordance with some embodiments.

As shown, the needle shaft 144 can include the one-or-more protrusions 230 configured to restrict the sheath 142 from sliding over the needle shaft 144 whether or not the sheath 142 includes the one-or-more through holes 232. The one-or-more protrusions 230 can be in the proximal portion of needle shaft 144 proximal of the needle slot 150 as shown in FIG. 15; however, the one-or-more protrusions 230 can alternatively be in the proximal portion of needle shaft 144 distal of the needle slot 150 or the distal portion of needle shaft 144 proximal of the needle tip 148. That said, the one-or-more protrusions 230 can alternatively be in some combination of the foregoing such as in the proximal portion of needle shaft 144 proximal of the needle slot 150 and distal of the needle slot 150. The one-or-more protrusions 230 can respectively be one or more pillars like those shown in FIG. 15 or one or more arcuate ridges, wherein each arcuate ridge of the one-or-more arcuate ridges is around at least a portion of a circumference of the needle shaft 144. That said, the one-or-more protrusions 230 can be a combination of pillars and arcuate ridges in embodiments of the needle shaft 144 including a plurality of the protrusions 230.

The sheath 142 can be disposed directly over the one-or-more protrusions 230 of the needle shaft 144, thereby fixing the sheath 142 to the needle shaft 144 by way of the one-or-more protrusions 230 protruding or biting into the sheath 142. Alternatively, the sheath 142 can include the one-or-more through holes 232 shaped in accordance with the one-or-more protrusions 230. When the sheath 142 is disposed over the needle shaft 144, the one-or-more protrusions 230 of the needle shaft 144 are disposed in the one-or-more through holes 232 of the sheath 142, respectively, thereby fixing the sheath 142 to the needle shaft 144.

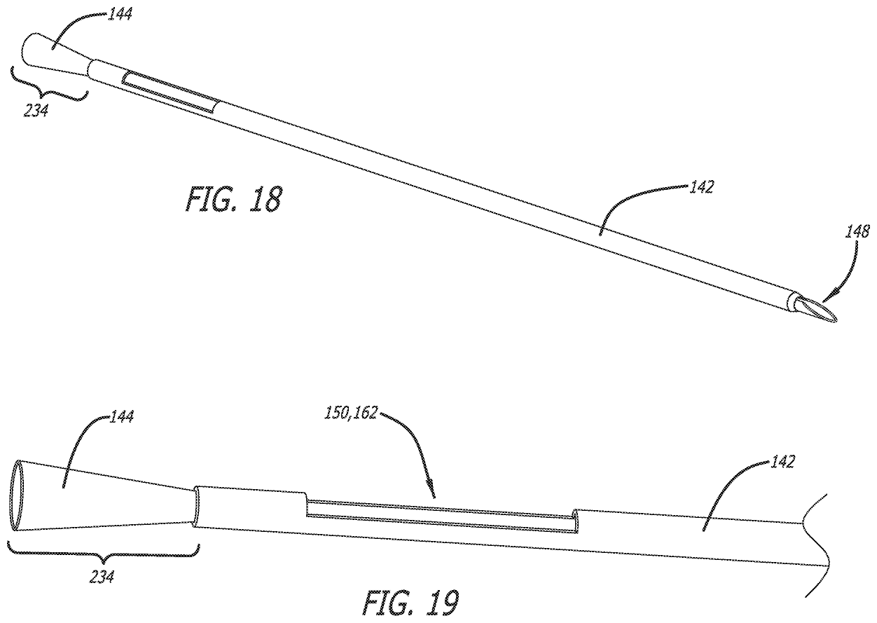

FIGS. 18 and 19 illustrate different views of the introducer needle 104 with the needle shaft 144 including a flared portion 234 in accordance with some embodiments.

As shown, the proximal portion of the needle shaft 144 proximal of the needle slot 150 can include the flared portion 234, thereby fixing the sheath 142 to the needle shaft 144 with respect to at least its proximal location on the needle shaft 144. Indeed, the flared portion 234 of the needle shaft 144 is configured to restrict the sheath 142 from proximally sliding over the needle shaft 144.

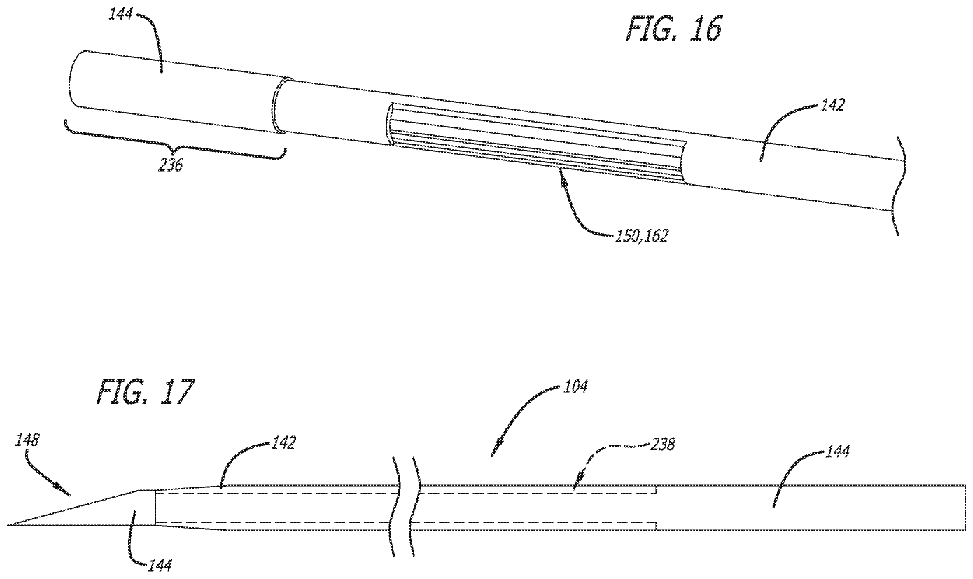

FIG. 16 illustrates a detailed view of the introducer needle 104 with the needle shaft 144 including a stepped portion 236 in accordance with some embodiments.

As shown, the proximal portion of the needle shaft 144 proximal of the needle slot 150 can include the stepped portion 236, thereby fixing the sheath 142 to the needle shaft 144 with respect to at least its proximal location on the needle shaft 144. Indeed, the stepped portion 236 of the needle shaft 144 is configured to restrict the sheath 142 from proximally sliding over the needle shaft 144.

FIG. 17 illustrates the introducer needle 104 with the needle shaft 144 including a recessed portion 238 in accordance with some embodiments.

As shown, the needle shaft 144 can include the recessed portion 238 between the proximal portion of the needle shaft 144 and the needle tip 148, thereby fixing the sheath 142 to the needle shaft 144 with respect to both its proximal and distal location on the needle shaft 144. Indeed, the recessed portion 238 of the needle shaft 144 is configured to restrict the sheath 142 from proximally or distally sliding over the needle shaft 144. Notably, the sheath 142 is disposed in the recessed portion 238 of the needle shaft 144 flush with a remainder of the needle shaft 144 so as to not catch on skin when a percutaneous puncture is performed with the introducer needle 104.

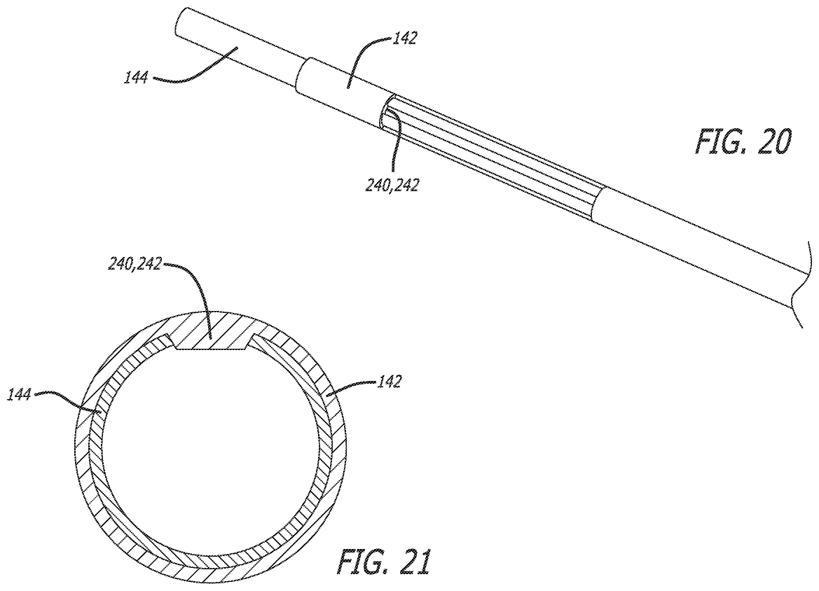

FIGS. 20 and 21 illustrate different views of the introducer needle 104 with the sheath 142 including an inwardly protruding protrusion 240 or longitudinal ridge 242 sitting in the needle slot 150 of the needle shaft 144 in accordance with some embodiments.

As shown, the sheath 142 can include the protrusion 240 in the proximal portion of sheath 142 configured sit within the needle slot 150 and abut a proximal end of the needle slot 150, thereby fixing the sheath 142 to the needle shaft 144 with respect to at least its proximal location on the needle shaft 144. Indeed, the protrusion 240 in combination with the proximal end of the needle slot 150 restricts the sheath 142 from proximally sliding over the needle shaft 144. Alternatively, the sheath 142 can include the ridge 242 extending from the proximal portion of sheath 142 to a distal portion of the sheath 142 (excluding the sheath opening 162). Like the protrusion 240, the ridge 242 is configured sit within the needle slot 150 and abut the proximal end of the needle slot 150 to restrict the sheath 142 from proximally sliding over the needle shaft 144.

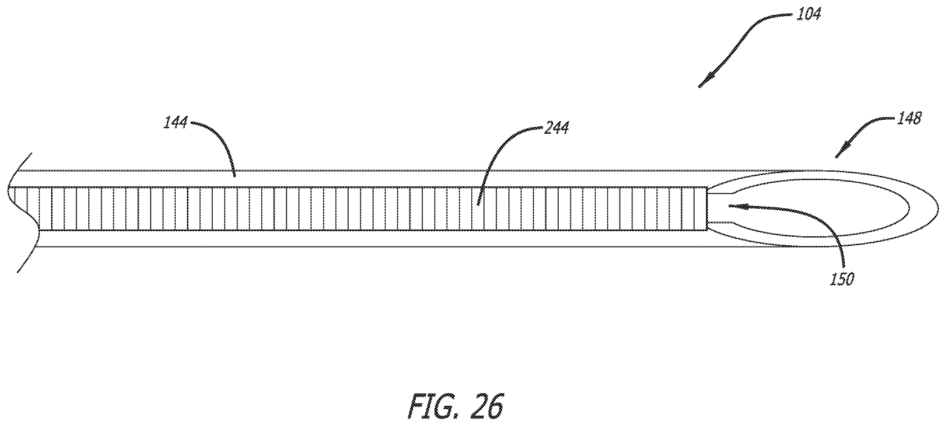

FIG. 26 illustrates a detailed view of the distal portion of the introducer needle 104 with a longitudinal strip 244 adhered to the needle shaft 144 over the needle slot 150 in accordance with some embodiments.

As shown, the introducer needle 104 need not include the sheath 142. Indeed, the introducer needle 104 can include the needle shaft 144, the strip 244 instead of the sheath 142, and the needle hub 146. As set forth herein, the needle shaft 144 includes the needle slot 150 extending from the proximal portion of the needle shaft 144 through the distal needle tip 148; however, in embodiments such as the instant embodiment, the strip 244—not the sheath 142—seals the needle slot 150 thereunder. Indeed, the strip 244 is adhered to the needle shaft 144 sealing the needle slot 150 thereunder but for a proximal portion of the needle slot 150, thereby creating an opening (not shown) in a side of the introducer needle 104 akin to the sheath opening 162. Like that set forth herein, the needle hub 146 is around at least the proximal portion of the needle shaft 144 in such embodiments.

The needle hub 146 includes an access-guidewire channel 164 in a distal portion of the needle hub 146 and a needle-hub connector in a proximal portion of the needle hub 146.

The access-guidewire channel 164 of the needle hub 146 is configured to allow the access guidewire 106 to pass over the needle hub 146 and direct the access guidewire 106 into the sealing module of the coupler 108. The access-guidewire channel 164 is open such that the access guidewire 106 lies in the access-guidewire channel 164 in at least the ready-to-operate state of the RICC insertion assembly 100. Advantageously, the open access-guidewire channel 164 allows the access guidewire 106 to remain in place when the introducer needle 104 is withdrawn from the RICC insertion assembly 100.

While not shown, the needle-hub connector includes a needle-hub bore and an optional needle-hub flange about the needle-hub connector.

The needle-hub bore of the needle-hub connector is configured to accept a syringe tip of the syringe 112 therein for fluidly connecting the introducer needle 104 to the syringe 112. Indeed, the needle-hub bore can have a Luer taper (e.g., a 6% taper) configured to accept the syringe tip therein, which syringe tip can be complementarily configured with a Luer taper.

The needle-hub flange of the needle-hub connector is configured to screw together with internal threads of a threaded collar around the syringe tip of the syringe 112. While the threaded collar of the syringe 112 is optional, the needle-hub flange advantageously provides a so-called Luer lock-style connection with the internal threads of the threaded collar when both are present. This provides added security against inadvertent disconnection of the introducer needle 104 and the syringe 112 over that provided by an otherwise Luer slip-style connection.

FIG. 1 illustrates the coupler 108 of the RICC insertion assembly 100 in accordance with some embodiments.

As shown, the coupler 108 includes a coupler housing 172, an access guidewire-connecting side arm 174, and, optionally, a splittable casing-holding side arm 176 when the RICC insertion assembly 100 also includes the keeper 220.

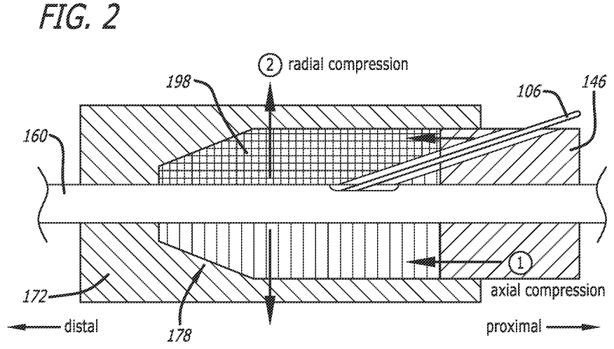

FIG. 2 illustrates a sealing-module cavity 178 and a needle-hub receptacle of the coupler housing 172 in accordance with some embodiments.

As shown, the coupler housing 172 includes the sealing-module cavity 178 and the needle-hub receptacle proximal of the sealing-module cavity 178. The coupler housing 172 also includes a longitudinal coupler-housing slot 182 formed along a length of the coupler housing 172 as shown in FIG. 1.

The sealing-module cavity 178 is configured to hold the sealing-module insert 198 therein as shown in FIG. 2.

The needle-hub receptacle is configured to hold the needle hub 146 of the introducer needle 104 therein as shown in FIG. 2. Indeed, the needle-hub receptacle includes the needle hub 146 inserted therein in the ready-to-operate state of the RICC insertion assembly 100. While not shown, the coupler 108 can include a needle-hub lock about the needle-hub receptacle configured to lock the needle hub 146 in the needle-hub receptacle. Indeed, a pair of lock buttons (e.g., spring-loaded lock buttons) of the needle-hub lock can be distributed between opposite sides of the coupler housing 172 such that each lock button of the lock buttons extends through the coupler housing 172 on its respective side of the coupler 108. Such lock buttons can be configured to unlock the needle hub 146 when the lock buttons are pressed into the coupler housing 172 for withdrawal of the introducer needle 104 from the coupler 108. Unlocking the lock buttons can immediately release the axial compression from the distal portion of the needle hub 146 compressing the sealing-module insert 198 in the sealing-module cavity 178 of the coupler housing 172. This allows the sealing-module insert 198 to relax for withdrawing the introducer needle 104 from the coupler 108 and, subsequently, the access guidewire 106.

The coupler-housing slot 182 is configured to allow the access guidewire 106 to escape from the coupler housing 172 after the introducer needle 104 is withdrawn from the coupler 108. Indeed, as the introducer needle 104 is withdrawn from the coupler 108, the sealing-module insert 198 releases the access guidewire 106 allowing it to escape from the coupler housing 172 by way of the coupler-housing slot 182.

Notably, the coupler housing 172 can be formed into a bullet-shaped body configured to be comfortably held underhand (e.g., cradled) or overhand in either a left hand for a left-handed percutaneous puncture (e.g., venipuncture) or a right hand for a right-handed percutaneous puncture (e.g., venipuncture) with the RICC insertion assembly 100. To further facilitate such venipunctures, an exterior of the coupler housing 172 can be textured with grip-enhancing ridges (e.g., transverse or circumferential ridges), protrusions, or the like.

The access guidewire-connecting side arm 174 extends from the coupler housing 172. While not shown, the access guidewire-connecting side arm 174 includes a connector configured to connect to the access-guidewire hub 218 about the proximal-end portion of the access guidewire 106, which access-guidewire hub 218 extends from the proximal end of the RICC 102 in at least the ready-to-operate state of the RICC insertion assembly 100. While in the ready-to-operate state of the RICC insertion assembly 100, the distal portion of the access guidewire 106 is disposed in the needle shaft 144 and the access-guidewire hub 218 is connected to the connector of the access guidewire-connecting side arm 174, thereby enforcing the loop 110 in the access guidewire 106 over which loop 110 the RICC 102 is disposed. When both the keeper 220 and the splittable casing-holding side arm 176 are present in the RICC insertion assembly 100, a distal portion of the splittable casing 222 is also held in the primary channel 190 of the splittable casing-holding side arm 176, thereby further enforcing the loop 110 in the access guidewire 106 over which loop 110 the RICC 102 is disposed.

When present, the splittable casing-holding side arm 176 extends from the coupler housing 172 opposite the access guidewire-connecting side arm 174. The splittable casing-holding side arm 176 includes a primary channel 190 and a secondary channel 192. The primary channel 190 is configured to slidably hold the splittable casing 222 or the longitudinal composite of the splittable casing 222 and at least the access guidewire 106 therein. The secondary channel 192 is configured to guide the access guidewire 106 split away from the splittable casing 222 into the coupler 108, the sealing module thereof, and the needle shaft 144 sealed therein by way of the needle slot 150. A divergent point 194 of the splittable casing-holding side arm 176 between the primary channel 190 and the secondary channel 192 is configured to split the access guidewire 106 away from the splittable casing 222 as the longitudinal composite of the splittable casing 222 and at least the access guidewire 106 therein is pushed therein.

FIG. 2 illustrates a sealing module of the coupler 108 of the RICC insertion assembly 100 in accordance with some embodiments.

The sealing module of the coupler 108 includes the sealing-module cavity 178 of the coupler housing 172 and an elastomeric (e.g., silicone) sealing-module insert 198 disposed therein. The sealing-module insert 198 includes a ‘Y’-shaped passageway therethrough configured to allow the access guidewire 106 to pass into the introducer needle 104 through a sidearm of the foregoing passageway. Indeed, the sidearm of the ‘Y’-shaped passageway is configured to direct the access guidewire 106 from the access-guidewire channel 164 of the needle hub 146 into both the sheath opening 162 of the sheath 142 and the needle slot 150 of the needle shaft 144 thereunder such that the access guidewire 106 can be disposed in the needle shaft 144 with the distal end of the access guidewire 106 just proximal of the needle tip 148 in the ready-to-operate state of the RICC insertion assembly 100. The sealing-module insert 198 is further configured to separately seal around the access guidewire 106 and the introducer needle 104 and when the sealing-module insert 198 is compressed in the sealing-module cavity 178 in the one-or-more states of the RICC insertion assembly 100 (e.g., the ready-to-operate state or the one-or-more operating states of the RICC insertion assembly 100).

The sealing module is configured to separately seal around the proximal portion of the introducer needle 104 and the distal portion of the access guidewire 106 when the sealing-module insert 198 is axially and radially compressed in the sealing-module cavity 178. For example, the sealing-module insert 198 can be both axially and radially compressed in the sealing-module cavity 178 by way of the distal portion of the needle hub 146. The distal portion of the needle hub 146 axially compresses the sealing-module insert 198 in the sealing-module cavity 178 when the needle hub 146 is disposed in the needle-hub receptacle such as in the ready-to-operate state of the RICC insertion assembly 100. Axial compression of the sealing-module insert 198 in the sealing-module cavity 178, in turn, radially compresses the sealing-module insert 198 in the sealing-module cavity 178, thereby sealing the sealing-module insert 198 around the introducer needle 104 and the access guidewire 106. The needle hub 146 can be removed from the needle-hub receptable in the one-or-more operating states of the RICC insertion assembly 100 to relieve both the axial and the radial compression and allow the introducer needle 104 to be withdrawn from the coupler 108 for the escape of the access guidewire 106 through the coupler-housing slot 182 thereafter.

FIG. 1 illustrates the access guidewire 106 disposed in the RICC insertion assembly 100 in accordance with some embodiments.

The access guidewire 106 includes a proximal portion including a proximal end and a distal portion including a distal end. In the ready-to-operate state of the RICC insertion assembly 100, the proximal end of the access guidewire 106 is coupled to the access guidewire-connecting side arm 174 by way of an access-guidewire hub 218 about a proximal-end portion of the access guidewire 106 that includes the distal end. In addition, the proximal portion of the access guidewire 106 extends along the primary lumen 130 of the RICC 102. The distal portion of the access guidewire 106 also extends along the primary lumen 130 of the RICC 102, but the distal portion of the access guidewire 106 further extends out the distal end of the RICC 102 as an extracatheteral portion of the access guidewire 106, into the sealing module over the needle hub 146 by way of the access-guidewire channel 164, into the needle shaft 144 through both the sheath opening 162 of the sheath 142 and the needle slot 150 of the needle shaft 144, and along the needle lumen 158 of the introducer needle 104 in the ready-to-operate state of the RICC insertion assembly 100. As shown in FIG. 1, the distal end of the access guidewire 106 is disposed in the needle lumen 158 just proximal of the needle tip 148 in the ready-to-operate state of the RICC insertion assembly 100. Again, the proximal and distal ends of the access guidewire 106 enforce the loop 110 in the access guidewire 106 in the ready-to-operate state of the RICC insertion assembly 100, which loop 110 the RICC 102 is disposed over, thereby keeping the RICC insertion assembly 100 in a relatively compact form.

The access guidewire 106 can include a guidewire tip in the distal portion of the access guidewire 106, which adopts a T shape configured to prevent puncturing a back wall of a blood vessel. Such a guidewire tip assumes a straightened state in the ready-to-operate state of the RICC insertion assembly 100 and a curved state when the guidewire tip is advanced beyond the needle tip 148 (e.g., advanced into a blood-vessel lumen) in the one-or-more operating states of the RICC insertion assembly 100 in which the access guidewire 106 is deployed.

The access guidewire 106 can further include a bare-wire portion and a wound-wire portion distal of the bare-wire portion, proximal of the bare-wire portion, or both. While not shown, the bare-wire portion, when present, distally extends through the sidearm of the ‘Y’-shaped passageway through the sealing module in at least the ready-to-operate state of the RICC insertion assembly 100 such that the sealing module forms a fluid-tight seal around the bare-wire portion of the access guidewire 106. Notably, the foregoing bare-wire portion can instead be a flat-wound or ground-wound portion of the access guidewire 106, wherein the flat-wound portion includes windings of a tape instead of a round wire, and wherein the ground-wound portion includes windings of a round wire ground down to flatten the windings.

FIG. 1 illustrate a keeper 220 of the RICC insertion assembly 100 in accordance with some embodiments.

As shown, the keeper 220 can include a splittable casing 222 and a catheter-hub holder 224 to which a proximal end of the splittable casing 222 is attached.

The splittable casing 222 can form a longitudinal composite with the catheter tube 114, the access guidewire 106, or both the catheter tube 114 and the access guidewire 106 in the RICC insertion assembly 100. With respect to the RICC insertion assembly 100 of FIG. 1, for example, the splittable casing 222 is over and, thusly, forms the longitudinal composite with both the catheter tube 114 and the access guidewire 106 in a portion of the RICC insertion assembly 100 nearest the catheter-hub holder 224, in which portion the access guidewire 106 is disposed in the primary lumen 130 of the RICC 102. Further with respect to the RICC insertion assembly 100 of FIG. 1, the splittable casing 222 is over and, thusly, forms the longitudinal composite with just the access guidewire 106 in a portion of the RICC insertion assembly 100 nearest the coupler 108 or the splittable casing-holding side arm 176 thereof, in which portion the extracatheteral portion of the access guidewire 106 extends from the distal end of the RICC 102. The splittable casing 222 is configured to split along its length, for example, as it is slid over the divergent point 194 of the splittable casing-holding side arm 176 of the coupler 108, such that the extracatheteral portion of the access guidewire 106 is initially revealed and the distal portion of the catheter tube 114 is subsequently revealed. In this way, the splittable casing 222 is configured to keep the catheter tube 114 and at least the distal portion of the access guidewire 106 sterile until deployed.

The catheter-hub holder 224 is configured to hold the catheter hub 116 therein as well as keep the splittable casing 222 in position over the catheter tube 114 and the access guidewire 106, particularly the extracatheteral portion of the access guidewire 106. The catheter-hub holder 224 includes a perimetrical wall 226 around at least a portion (e.g., a proximal portion) of a perimeter of the catheter-hub holder 224. The perimetrical wall 226 defines a recess into which the catheter hub 116 fits with an engineering fit (e.g., a clearance fit such as a running, sliding, or location fit or a transition fit such as a similar or fixed fit as classified by the ISO as well as one or more gaps for extension of the one-or-more extension legs 118 therethrough. Additionally or alternatively, the catheter-hub holder 224 can include a wing corresponding to a suture wing of the catheter hub 116. Such a wing can include posts configured to insert into suture-wing holes of the suture wing of the catheter hub 116 with an engineering fit.

### Methods

Methods include at least a method of making the introducer needle 104. Such a method includes one or more steps selected from a metal strip-rolling step, a seam-welding step, a cold-working step, a grinding step, a needle-slot creating step, an edge-finishing step, a sheath-disposing step, a sheath-fixing step, a sheath opening-creating step, and a needle hub-fixing step.

The metal strip-rolling step includes rolling a strip of metal such as stainless steel into a metal tube. For example, the metal strip-rolling step can include rolling the strip of metal into the metal tube with a milling machine. Such a metal tube includes a longitudinal seam formed between edges of longitudinal sides of the strip of metal.

The seam-welding step includes welding the seam formed between the edges of the sides of the strip of metal. For example, the seam-welding step can include laser welding the seam formed between the edges of the sides of the strip of metal.

The cold-working step includes pushing the metal tube through one or more dies one or more times, thereby reducing an outer diameter of the metal tube while simultaneously increasing a thickness of a metal-tube wall of the metal tube. Subsequent to the cold-working step, the metal tube can be scored and broken into two or more smaller metal tubes and batched together for the grinding step; however, for expository convenience, such further processing is described with reference to the foregoing metal tube.

The grinding step includes grinding an end of the metal tube at a plurality of angles to form the needle shaft 144 with the needle tip 148 having the bevel. For example, grinding the end of the metal tube at a first angle forms the primary bevel 154 including the heel of the bevel; grinding the end of the metal tube at two similar, but opposite second angles forms the tip bevel 152 of the bevel.

The needle-slot creating step includes creating the needle slot 150 in the needle shaft 144. In an example, the needle-slot creating step can include cutting the needle slot 150 into the needle shaft 144 by way of machining or laser cutting. Needle-slot walls that result from the cutting of the needle slot 150 into the needle shaft 144 face each other and are parallel to each other. In another example, the needle-slot creating step can include grinding the needle slot 150 into the needle shaft 144. The needle-slot walls that result from the grinding of the needle slot 150 into the needle shaft 144 face away from a bottom of the needle slot 150. As set forth above, the needle slot 150 extends from the proximal portion of the needle shaft 144 through the needle tip 148.

The edge-finishing step includes finishing (e.g., grinding, polishing, etc.) edges of the needle-slot walls, thereby minimizing or eliminating sharp, access guidewire-fraying edges of the needle slot 150.

The sheath-disposing step includes disposing the sheath 142 over the needle shaft 144, which sheath 142 seals the needle slot 150 thereunder. The disposing of the sheath 142 over the needle shaft 144 can include inserting the needle shaft 144 into the sheath 142.

The sheath-fixing step includes any of a number of ways of fixing the sheath 142 to the needle shaft 144 in accordance with embodiments of the introducer needle 104 set forth above. In an example, the sheath-fixing step can include heat shrinking the sheath 142 over the needle shaft 144, which can result in the engineering fit selected from the transition fit and the interference fit. In another example, the sheath-fixing step can include adhering the sheath 142 to the needle shaft 144 with the adhesive 228 selected from the pressure-sensitive adhesive, the epoxy, and the tie-layer resin. In another example, the sheath-fixing step can include laser-welding the sheath 142 to the needle shaft 144. Again, such a sheath 142 is translucent to at least the wavelength or range of wavelengths of the laser used for the laser-welding. In another example, the sheath-fixing step can include welding (e.g., microwelding, laser welding, etc.) the one-or-more protrusions 230 onto the needle shaft 144, cutting the one-or-more through holes 232 into the sheath 142, and disposing the sheath 142 over the needle shaft 144 such that the one-or-more protrusions 230 are disposed in the one-or-more through holes 232, respectively. In another example, the sheath-fixing step can include recessing a portion of the needle shaft 144 between the proximal portion of the needle shaft 144 and the needle tip 148 to create the recessed portion 238 of the needle shaft 144 and subsequently disposing the sheath 142 over the needle shaft 144 in the recessed portion 238 of the needle shaft 144 such that the sheath 142 is flush with a remainder of the needle shaft 144.

The sheath opening-creating step includes creating the sheath opening 162 in the sheath 142. The creating of the sheath opening 162 in the sheath 142 can include cutting (e.g., laser cutting) the sheath opening 162 into the needle shaft 144 before or after the disposing of the sheath 142 over the needle shaft 144 in the sheath-disposing step. As set forth above, the sheath 142 seals the needle slot 150 thereunder but for the sheath opening 162 in the proximal portion of the sheath 142.

The needle hub-fixing step includes fixing the needle hub 146 around at least the proximal portion of the needle shaft 144, thereby forming the introducer needle 104. The needle hub-fixing step can include pressing the proximal portion of the needle shaft 144 into the needle hub 146 with an engineering fit selected from a transition and an interference fit. Additionally or alternatively, the needle hub-fixing step can include adhering the needle hub 146 to the proximal portion of the needle shaft 144.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. An introducer needle, comprising:
a needle shaft including a needle slot extending from a proximal portion of the needle shaft through a distal needle tip;
a sheath disposed over the needle shaft and fixed in at least location thereon, the sheath sealing the needle slot thereunder but for a sheath opening in a proximal portion of the sheath; and
a needle hub around at least the proximal portion of the needle shaft.

2. The introducer needle of claim 1, wherein the sheath is adhered to the needle shaft with an intervening adhesive.

3. The introducer needle of claim 2, wherein the adhesive is a pressure-sensitive adhesive.

4. The introducer needle of claim 2, wherein the adhesive is a cured epoxy resin.

5. The introducer needle of claim 2, wherein the adhesive is a cured tie-layer resin.

6. The introducer needle of claim 1, wherein the sheath is laser-welded to the needle shaft, the sheath translucent to at least a wavelength or range of wavelengths of a laser used to laser-weld the sheath to the needle shaft.

7. The introducer needle of claim 6, wherein the sheath is translucent to visible light, thereby enabling a user to witness blood flash into the needle shaft through the sheath sealing the needle slot thereunder upon performing a percutaneous puncture with the introducer needle.

8. The introducer needle of claim 1, wherein the needle shaft includes one or more outwardly protruding protrusions in the proximal portion of needle shaft proximal of the needle slot, the proximal portion of needle shaft distal of the needle slot, a distal portion of needle shaft proximal of the needle tip, or a combination thereof, the one-or-more protrusions configured to restrict the sheath from sliding over the needle shaft.

9. The introducer needle of claim 8, wherein the one-or-more protrusions are one or more pillars, respectively.

10. The introducer needle of claim 8, wherein the one-or-more protrusions are one or more arcuate ridges, respectively, each arcuate ridge of the one-or-more arcuate ridges along at least a portion of a circumference of the needle shaft.

11. The introducer needle of claim 8, wherein the sheath includes one or more through holes shaped in accordance with the one-or-more protrusions, the one-or-more protrusions of the needle shaft disposed in the one-or-more through holes of the sheath, respectively.

12. The introducer needle of claim 1, wherein the proximal portion of the needle shaft proximal of the needle slot includes a flared portion configured to restrict the sheath from proximally sliding over the needle shaft.

13. The introducer needle of claim 1, wherein the proximal portion of the needle shaft proximal of the needle slot includes a stepped portion configured to restrict the sheath from proximally sliding over the needle shaft.

14. The introducer needle of claim 1, wherein the needle shaft includes a recessed portion between the proximal portion of the needle shaft and the needle tip configured to restrict the sheath from proximally or distally sliding over the needle shaft, the sheath disposed in the recessed portion of the needle shaft flush with a remainder of the needle shaft.

15. The introducer needle of claim 1, wherein the sheath includes an inwardly protruding protrusion in the proximal portion of sheath configured sit within the needle slot and abut a proximal end of the needle slot to restrict the sheath from proximally sliding over the needle shaft.

16. The introducer needle of claim 1, wherein the sheath includes an inwardly protruding longitudinal ridge extending from the proximal portion of sheath to a distal portion of the sheath, the ridge configured sit within the needle slot and abut a proximal end of the needle slot to restrict the sheath from proximally sliding over the needle shaft.

17. The introducer needle of claim 1, wherein the sheath is disposed over the needle shaft with an engineering fit selected from a transition fit and an interference fit.

18. The introducer needle of claim 1, wherein an outer surface of the needle shaft is textured or roughened.

19. The introducer needle of claim 1, wherein the sheath has a sheath-wall thickness from about 0.001″ to about 0.003″.

20. The introducer needle of claim 1, wherein the sheath has a sheath-wall thickness of about 0.003″ to about 0.006″.

21. The introducer needle of claim 1, wherein the sheath has a sheath-wall thickness from about 0.006″ to about 0.008″.

22. An introducer needle, comprising:
a needle shaft including a needle slot extending from a proximal portion of the needle shaft through a distal needle tip;
a longitudinal strip adhered to the needle shaft, the strip sealing the needle slot thereunder but for a proximal portion of the needle slot, thereby creating an opening in a side of the introducer needle; and
a needle hub around at least the proximal portion of the needle shaft.

23-30. (canceled)
